(19) United States
(12) Patent Application Publication
(10) Pub. No.: US 2015/0218145 A1
(43) Pub. Date: Aug. 6, 2015

(54) PROCESS FOR THE PREPARATION OF RIVAROXABAN

(71) Applicant: RANBAXY LABORATORIES LIMITED, New Delhi, Delhi (IN)

(72) Inventors: Mohammed Salman Hashmi, Aligarh (IN); Yoginder Pal Sachdeva, Firozepur (IN)

(21) Appl. No.: 14/430,279

(22) PCT Filed: Sep. 26, 2013
(86) PCT No.: PCT/IB2013/058897

§ 371 Date: Mar. 23, 2015

(30) Foreign Application Priority Data
Sep. 26, 2012 (IN) 3005/DEL/2012

(51) Int. Cl. C07D 413/14

(52) U.S. Cl. CPC C07D 413/14

(57) ABSTRACT

The present invention provides a process for the preparation of rivaroxaban in a process that includes cyclizing a compound of Formula (II) with a dialkyl carbonate in the presence of a base.

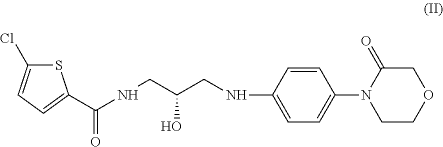 [SMILES: O=C(NC[C@@H](O)CNc1ccc(N2CCOCC2=O)cc1)c1ccc(Cl)s1]

### FIELD OF THE INVENTION

The present invention provides a process for the preparation of rivaroxaban.

### BACKGROUND OF THE INVENTION

Rivaroxaban chemically is 5-chloro-N-({(5S)-2-oxo-3-[4-(3-oxo-4-morpholinyl) phenyl]-1,3-oxazolidin-5-yl}methyl)-2-thiophenecarboxamide of Formula I.

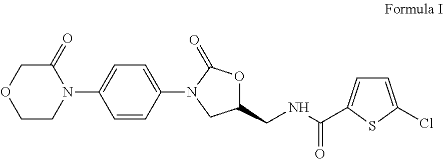

Rivaroxaban is used as an anti-thrombotic agent.

U.S. Pat. No. 7,157,456 provides rivaroxaban and processes for its preparation. Example 44 of the '456 Patent provides a process of making rivaroxaban. However, Example 44 does not disclose a step of cyclizing a compound of Formula II with a dialkyl carbonate to produce the compound of Formula I.

U.S. Pat. No. 8,106,192 provides a process for the preparation of rivaroxaban, wherein N-{(R)-2-hydroxy-3-[4-(3-oxomorpholin-4-yl)phenylamino]propyl}-5-chlorothiophene-2-carboxamide is treated with N,N-carbonyldiimidazole in the presence of 1-methyl-2-pyrrolidone and toluene. N,N-Carbonyldiimidazole is costly, toxic, moisture sensitive, and may produce toxic by-products during the reaction.

The prior art processes for the preparation of rivaroxaban make use of chromatography or reagents that are costly, unstable, and have safety concerns. Accordingly, these processes are not suitable at industrial scale.

The present inventors have developed a simple, safe, efficient, economical, industrially feasible process that provides rivaroxaban in good yield.

### SUMMARY OF THE INVENTION

The present invention provides processes for the preparation of rivaroxaban.

In one general aspect, a process for the preparation of rivaroxaban of Formula I

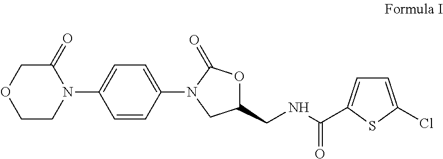

Includes a step of cyclizing a compound of Formula II

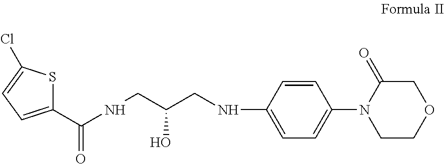 [SMILES: O=C(NC[C@@H](O)CNc1ccc(N2CCOCC2=O)cc1)c1ccc(Cl)s1]

with a dialkyl carbonate.

Embodiments of the process may include one or more of the following features. For example, the compound of Formula II may be cyclized with the dialkyl carbonate in the presence of a base. The dialkyl carbonate may be selected from one or more of dimethyl carbonate, diethyl carbonate, dipropyl carbonate, diisopropyl carbonate, dibutyl carbonate, or diisobutyl carbonate. The base may be selected from sodium carbonate, potassium carbonate, calcium carbonate, sodium hydroxide, potassium hydroxide, or a mixture thereof.

In the process, the base may be added to a solution of N-{(R)-2-hydroxy-3-[4-(3-oxomorpholin-4-yl)phenylamino]propyl}-5-chlorothiophene-2-carboxamide (Formula II) in the alkyl carbonate and the mixture refluxed.

The process may further include the steps of (a) recovering the solvent under vacuum at 60° C. to 65° C.; (b) treating the solid material obtained with dichloromethane; and (c) filtering the solid material to remove any inorganic salt. The process may still further include (a) recovering the solvent under vacuum; and (b) crystallizing the material obtained from dichloromethane.

In the process, the alkyl groups in the dialkyl carbonate may be the same or different.

In another general aspect, the invention relates to a process for the preparation of rivaroxaban of Formula I

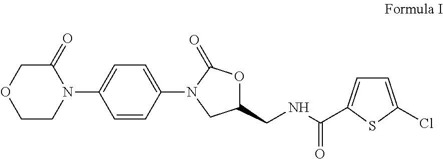

using a process that includes cyclizing a compound of Formula II

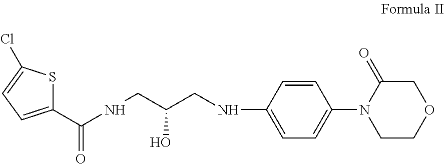 [SMILES: O=C(NC[C@@H](O)CNc1ccc(N2CCOCC2=O)cc1)c1ccc(Cl)s1]

with a dialkyl carbonate in the presence of a base. The process includes the steps of

adding the base to a solution of N-{(R)-2-hydroxy-3-[4-(3-oxomorpholin-4-yl) phenylamino]propyl}-5-chlorothiophene-2-carboxamide (Formula II) in the alkyl carbonate and refluxing the resultant mixture;

recovering the solvent under vacuum;

treating the solid material obtained with dichloromethane;

filtering the solid material to remove any inorganic salt;

recovering the solvent under vacuum; and

crystallizing the material obtained from dichloromethane.

Embodiments of the process may include one or more of the following steps. For example, the dialkyl carbonate may be selected from one or more of dimethyl carbonate, diethyl carbonate, dipropyl carbonate, diisopropyl carbonate, dibutyl carbonate, or diisobutyl carbonate. The base may be selected from sodium carbonate, potassium carbonate, calcium carbonate, sodium hydroxide, potassium hydroxide, or a mixture thereof.

In the process, the solvent may be recovered under vacuum at a temperature of 60° C. to 65° C.

In the process, the alkyl groups in the dialkyl carbonate may be the same or different.

### DETAILED DESCRIPTION OF THE INVENTION

A first aspect of the present invention provides a process for the preparation of rivaroxaban of Formula I

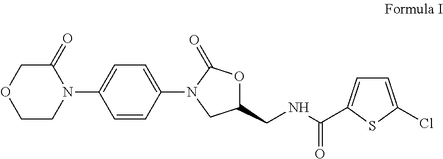

wherein the process comprises cyclizing a compound of Formula II

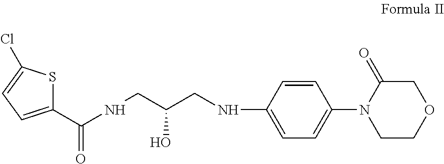 [SMILES: O=C(NC[C@@H](O)CNc1ccc(N2CCOCC2=O)cc1)c1ccc(Cl)s1]

with a dialkyl carbonate.

A second aspect of the present invention provides a process for the preparation of rivaroxaban of Formula I

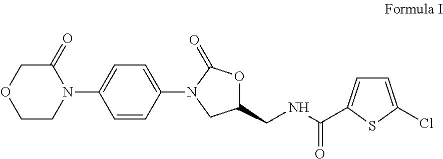

wherein the process comprises cyclizing a compound of Formula II

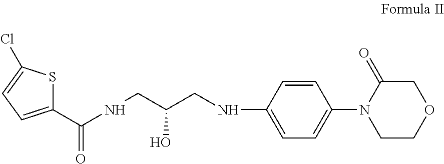 [SMILES: O=C(NC[C@@H](O)CNc1ccc(N2CCOCC2=O)cc1)c1ccc(Cl)s1]

with dimethyl carbonate, diethyl carbonate, dipropyl carbonate, diisopropyl carbonate, dibutyl carbonate, or diisobutyl carbonate.

A third aspect of the present invention provides a process for the preparation of rivaroxaban of Formula I

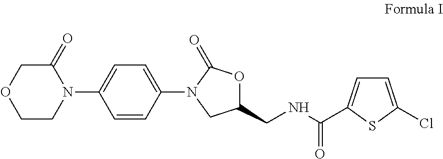

wherein the process comprises cyclizing a compound of Formula II

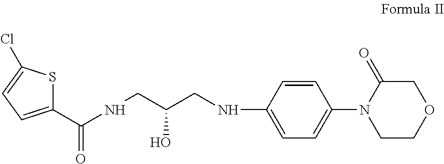 [SMILES: O=C(NC[C@@H](O)CNc1ccc(N2CCOCC2=O)cc1)c1ccc(Cl)s1]

with a diethyl carbonate.

The compound of Formula II may be prepared according to the process provided in the art, for example, the process described in U.S. Pat. No. 8,106,192. The compound of Formula II is cyclized with a dialkyl carbonate, optionally in the presence of a base. The dialkyl carbonate may be, for example, dimethyl carbonate, diethyl carbonate, dipropyl carbonate, diisopropyl carbonate, dibutyl carbonate, or diisobutyl carbonate or combinations of dialkyl carbonates. It is expected that other dialkyl carbonates in which the alkyl groups are not the same will also function in the above reaction to cyclize the compound of Formula II. Therefore in one general aspect, the dialkyl carbonate is a compound in which the alkyl groups in the dialkyl carbonate are the same. In another general aspect, the alkyl groups in the dialkyl carbonate are not the same. The base may be, for example, sodium carbonate, potassium carbonate, calcium carbonate, sodium hydroxide, potassium hydroxide, or a mixture thereof. The compound of Formula II may be heated with dialkyl carbonate for about 1 hour to about 8 hours above the boiling point of the alkanol produced during the reaction. The product may be isolated from the mixture by methods including concentration, distillation, decantation, filtration, evaporation, centrifugation, or a combination thereof, and may further be crystallized.

The term “dialkyl carbonate”, as used herein, refers to a carbonate group flanked by two alkyl groups.

The term “alkyl”, as used herein, refers to saturated, aliphatic hydrocarbon groups, either straight or branched-chain, containing from one to four carbon atoms, as exemplified by methyl, ethyl, propyl, isopropyl, butyl, or isobutyl.

The term “alkanol”, as used herein, refers to an “alkyl” as defined above containing at least one hydroxyl group.

The term “about”, as used herein, when used along with values assigned to certain measurements and parameters means a variation of up to 10% from such values, or in case of a range of values, means up to a 10% variation from both the lower and upper limits of such ranges.

While the present invention has been described in terms of its specific embodiments, certain modifications and equivalents will be apparent to those skilled in the art, and are intended to be included within the scope of the present invention.

### EXAMPLE

### Preparation of 5-Chloro-N-({(5S)-2-Oxo-3-[4-(3-Oxo-4-Morpholinyl) Phenyl]-1,3-Oxazolidin-5-Yl}Methyl)-2-Thiophenecarboxamide (Formula I)

Potassium carbonate (0.64 g; 4.6 mmoles) was added to a solution of N-{(R)-2-hydroxy-3-[4-(3-oxomorpholin-4-yl) phenylamino]propyl}-5-chlorothiophene-2-carboxamide (Formula II; 1 g; 2.24 mmoles) in diethyl carbonate (4 mL). The mixture was refluxed for 4 hours. The solvent was recovered under vacuum at 60° C. to 65° C. The solid material obtained was treated with dichloromethane (10 mL) and filtered to remove the inorganic salt. The solvent was recovered under vacuum at 35° C. and the material obtained was crystallized from dichloromethane (5 mL). Yield =0.9 g (85%).

It should be understood that variations in the above processes are contemplated. For example, the diethyl carbonate may be replaced by any of dimethyl carbonate, dipropyl carbonate, diisopropyl carbonate, dibutyl carbonate, diisobutyl carbonate or other dialkyl carbonate. Similarly, the base used in the example, potassium carbonate, may be replaced by sodium carbonate, calcium carbonate, sodium hydroxide, potassium hydroxide, or a mixture thereof.

1. A process for the preparation of rivaroxaban of Formula I 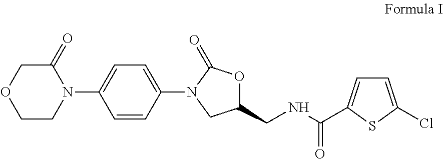
wherein the process comprises cyclizing a compound of Formula II 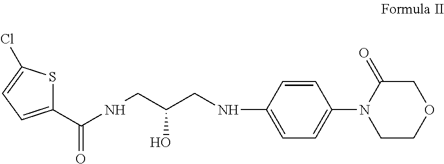 [SMILES: O=C(NC[C@@H](O)CNc1ccc(N2CCOCC2=O)cc1)c1ccc(Cl)s1]
with a dialkyl carbonate.

2. The process according to claim 1, wherein the compound of Formula II is cyclized with the dialkyl carbonate in the presence of a base.

3. The process according to claim 1, wherein the dialkyl carbonate is selected from one or more of dimethyl carbonate, diethyl carbonate, dipropyl carbonate, diisopropyl carbonate, dibutyl carbonate, or diisobutyl carbonate.

4. The process according to claim 2, wherein the dialkyl carbonate is selected from one or more of dimethyl carbonate, diethyl carbonate, dipropyl carbonate, diisopropyl carbonate, dibutyl carbonate, or diisobutyl carbonate.

5. The process according to claim 2, wherein the base is selected from sodium carbonate, potassium carbonate, calcium carbonate, sodium hydroxide, potassium hydroxide, or a mixture thereof.

6. The process according to claim 2, wherein the base is added to a solution of N-{(R)-2-hydroxy-3[4-(3-oxomorpholin-4-yl) phenylamino]propyl}-5-chlorothiophene-2-carboxamide (Formula II) in the dialkyl carbonate and the mixture refluxed.

7. The process according to claim 6, further comprising:
recovering the solvent under vacuum at 60° C. to 65° C.;
treating the solid material obtained with dichloromethane; and
filtering the solid material to remove any inorganic salt.

8. The process according to claim 7, further comprising:
recovering the solvent under vacuum; and
crystallizing the material obtained from dichloromethane.

9. The process according to claim 1, wherein the alkyl groups in the dialkyl carbonate are the same.

10. A process for the preparation of rivaroxaban of Formula I 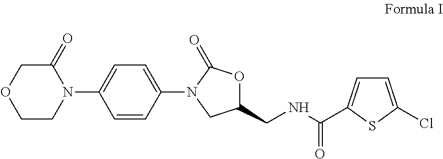
wherein the process comprises cyclizing a compound of Formula II 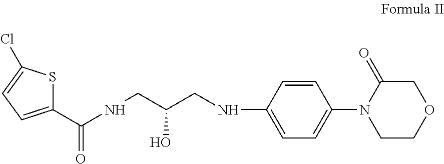 [SMILES: O=C(NC[C@@H](O)CNc1ccc(N2CCOCC2=O)cc1)c1ccc(Cl)s1]
with a dialkyl carbonate in the presence of a base, the method comprising:
adding the base to a solution of N-{(R)-2-hydroxy-3-[4-(3-oxomorpholin-4-yl) phenylamino]propyl}-5-chlorothiophene-2-carboxamide (Formula II) in the dialkyl carbonate and refluxing the resultant mixture;
recovering the solvent under vacuum;
treating the solid material obtained with dichloromethane;
filtering the solid material to remove any inorganic salt;
recovering the solvent under vacuum; and
crystallizing the material obtained from dichloromethane.

11. The process according to claim 10, wherein the dialkyl carbonate is selected from one or more of dimethyl carbonate, diethyl carbonate, dipropyl carbonate, diisopropyl carbonate, dibutyl carbonate, or diisobutyl carbonate.

12. The process according to claim 10, wherein the base is selected from sodium carbonate, potassium carbonate, calcium carbonate, sodium hydroxide, potassium hydroxide, or a mixture thereof.

13. The process according to claim 10, wherein the solvent is recovered under vacuum at 60° C. to 65° C.

14. The process according to claim 10, wherein the alkyl groups in the dialkyl carbonate are the same.
